(19) United States
(12) Patent Application Publication
(10) Pub. No.: US 2009/0118225 A1
(43) Pub. Date: May 7, 2009

(54) 1-Methyl Nicotinamide and Derivatives for Treatment of Gastric Injury

(75) Inventors: Alexander Krantz, Boston, MA (US); Tomasz Brzozowski, Cracow (PL)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Cortria Corporation, Boston, MA (US)

(21) Appl. No.: 12/210,952

(22) Filed: Sep. 15, 2008

Related U.S. Application Data
(60) Provisional application No. 60/995,765, filed on Sep. 28, 2007.

(51) Int. Cl. A61K 31/737; A61K 31/455; A61K 31/727; A61P 1/04

(52) U.S. Cl. 514/53; 514/355; 514/356; 514/56

(57) ABSTRACT

The present invention is directed to nicotinamide derivatives, and their use in treating gastrointestinal disorders.

### CROSS REFERENCE TO RELATED APPLICATIONS

Application claims priority under 35 U.S.C. § 119(e) to U.S. Provisional Application No. 60/995,765, filed on Sep. 28, 2007, which application is incorporated by reference herein in its entirety.

### BACKGROUND OF THE INVENTION

Gastrointestinal problems are common. Most people have experienced some form of gastrointestinal upset in their lives: nausea, vomiting or diarrhea associated with the flu, or indigestion after eating excessively. Over 95 million Americans have experienced gastrointestinal upset, and over 10 million Americans are hospitalized each year for care of gastrointestinal problems. While many digestive problems are more common as people get older, they can occur at any age, even in children, and strike regardless of gender, ethnic or socioeconomic backgrounds. Heartburn and ulcers are well known examples of common gastrointestinal problems. These disorders result from and are exacerbated by excessive secretion of gastric acid in the digestive tract.

Heartburn, also known as acid indigestion or Gastro-Esophageal Reflux Disease (GERD), is caused by the backward flow of acid from the stomach up into the esophagus. People with heartburn experience burning chest pain localized behind the breastbone that moves up toward the neck and throat. Some even experience a bitter or sour taste of acid in the back of the throat. The burning and pressure symptoms of heartburn can last as long as 2 hours and are often worsened by eating food. At least 60 million Americans experience heartburn at least once a month, and some studies have suggested that over 15 million Americans experience heartburn daily.

Peptic ulcers represent a major health problem, both in terms of morbidity and mortality. Research advances during the last decade have offered new insights in the therapy and prevention of gastroduodenal ulceration by measures directed at strengthening the mucosal defense system rather than by attenuating the aggressive acid-pepsin factors held responsible for the induction of ulcers. The rise in gastric acidity and peptic activity are usually a manifestation of a physiological disturbance affecting one or more mechanisms that normally regulate gastric secretion.

Neurotransmitters or hormones that directly stimulate secretion of hydrochloric acid and pepsin by the gastric glands are acetylcholine, gastrin and histamine. In addition, there are other factors that play an important role in the manifestation of peptic ulcers. Activity of the gastric secretary cells has been found to be stimulated by caffeine, alcohol, hydrochloric acid, sodium chloride, non steroidal anti-inflammatory drugs (NSAIDS) and stress.

NSAIDs, such as acetylsalicylic acid (ASA), diclofenac, indomethacin, ibuprofen and naproxen, are widely used in the clinic. From a pharmacological point of view they act as inhibitors of the cyclooxygenase (COX). The anti-inflammatory properties of NSAIDs are related to their suppression of prostaglandin synthesis. However, suppression of gastric prostaglandins decreases gastric mucosal blood flow, with concomitant mucosal sensitivity to topical injury by a variety of irritants. Gastric ulceration induced by NSAIDs significantly limits the utility of these drugs.

“Ulcer disease,” which is a more accurate designation than “peptic ulcer,” is a mass disorder because, just as in cardiovascular diseases and cancer, it affects a large segment of the population, and its mechanism(s) of development is (are) poorly understood. It is now clear that ulcer disease is a complex disorder that is multifactorial and pluricausal in origin. The multifactorial etiology and pathogenesis imply that it is unrealistic to expect a complete healing or a preventive effect from highly specific drugs that affect only one component in this complex chain of events. It is thus not surprising that, after cessation of treatment with even the currently most potent antisecretory agent (the H₂-receptor antagonists such as cimetidine), the recurrence rate of chronic duodenal ulcers is 40-60% a year (Thomas et al., 1984 Clin. Gastroenterol. 13: 501-54). Novel drugs that affect more than one element in the pathogenesis of ulcer disease are thus realistically expected to have a more profound effect on ulcer healing and recurrence than presently available anti-ulcer drugs.

Medications for treating gastrointestinal disorders relating to excessive acid secretion are currently available. However, these medications fail to alleviate symptoms in a significant number of patients, up to 50% of patients for certain classes of medications. Such patients continue to experience gastrointestinal discomfort that many would agree has reduced their quality of life.

Most of the symptoms experienced by patients under such conditions result from a breakdown of the normal mucosal defense mechanisms. Various studies have demonstrated that gastric acid and pepsin are important in the pathogenesis of dyspepsia, stomach upset, gastro-esophageal reflux disease, and duodenal and gastric ulcer. Several mechanisms are believed to be important in protecting gastric and duodenal mucosa from damage by gastric acid, pepsin, bile pancreatic enzymes, as well as these external stressors/factors. These defense mechanisms include mucus, mucosal blood flow, cell renewal and bicarbonate. These factors acting in balance help maintain mucosal integrity.

Nicotinamide N-methyltransferase (EC 2.1.1.1, NNMT) catalyzes the transfer of a methyl group from S-adenosylmethionine to nicotinamide (NA) to produce 1-methyl-nicotinamide (1-MNA) and S-adenosylhomocysteine (SAH). The enzyme is known to be inhibited by elevated levels of its products, 1-MNA or SAH (Aksoy et al., 1994 J. Biol. Chem. 269: 14835-40.)

1-MNA has been linked to diabetes and other disease states (Gosteli, J., 2005 Med. Hypotheses 64: 1062-3, and Gebicki et al., 2003 Pol. J. Pharmacol. 55: 109-12). Nicotinamide has a long history in the treatment of disease as a form of vitamin B3 and has been evaluated in clinical trials for the treatment of diabetes.

Recently, nicotinamide was determined to be a noncompetitive inhibitor of a Sirtuin enzyme, a member of a unique class of NAD(+)-dependent deacetylases required for diverse biological processes, including transcriptional silencing, regulation of apoptosis, fat mobilization, and lifespan regulation (Avalos et al., 2005 Mol. Cell. 17: 855-68.)

It is well known that nicotinic acid (NAc) in high doses possesses important properties in the correction of lipoprotein profile (i.e. the treatment of lipoprotein abnormalities), mostly by reducing triglyceride (TG) and LDL-cholesterol levels as well as elevating HDL levels. The main disadvantage of nicotinic acid therapy is associated with its side effects.

Current treatments for gastric discomfort include administration of antacids and H₂-receptor antagonists. However, these treatments are not effective in preventing gastric injury over the long term. There remains a need for an effective method to prevent and/or treat gastric injury in persons who have or are at risk for such injury. The present invention fulfills this need, and provides further related advantages.

### BRIEF SUMMARY OF THE INVENTION

The invention is directed to a pharmaceutical composition comprising a compound of formula (I):

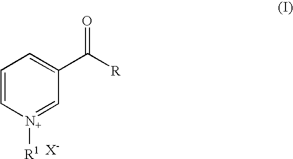

wherein

R is the group NR²R³ or the group OR⁴;

R¹ is methyl;

R² and R⁴ each independently is hydrogen or C₁₋₄alkyl;

R³ is hydrogen, C₁₋₄alkyl or CH₂OH; and

X⁻ is a physiologically suitable counter-anion.

In one embodiment, R is the group NR²R³. In another embodiment, R² is methyl or hydrogen. In still another embodiment, R³ is CH₂OH or hydrogen.

In another embodiment, R is the group OR⁴, and R⁴ is C₁₋₄ alkyl. In yet another embodiment, R⁴ is propyl or ethyl.

In another embodiment, the compound of formula (I) is selected from a 1-methylnicotinamide salt or a 1-methyl-N′-hydroxymethylnicotinamide salt. In yet another embodiment, the compound of formula (I) is selected from a 1-methylnicotinic acid ethyl ester salt or a 1-methylnicotinic acid propyl ester salt. In another embodiment, the compound of formula (I) is selected from a 1-methylnicotinic acid salt. In another embodiment, the salt is a chloride, benzoate, salicylate, acetate, citrate or lactate. In another embodiment, the compound of formula (I) is selected from 1-methylnicotinamide chloride, 1-methylnicotinamide citrate, 1-methylnicotinamide lactate, 1-methyl-N′-hydroxymethylnicotinamide chloride, 1-methylnicotinic acid chloride, 1-methylnicotinic acid ethyl ester chloride or 1-methylnicotinic acid propyl ester chloride.

The invention includes a method of treating a gastrointestinal disorder in a subject in need thereof. The method comprises administering to the subject a pharmaceutical composition comprising a compound of formula (I):

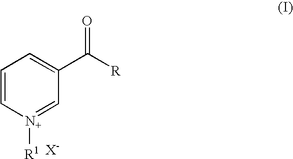

wherein

R is the group NR²R³ or the group OR⁴;

R¹ is methyl;

R² and R⁴ each independently is hydrogen or C₁₋₄alkyl;

R³ is hydrogen, C₁₋₄alkyl or CH₂OH; and

X⁻ is a physiologically suitable counter-anion.

In one aspect, R is the group NR²R³. In another aspect, R² is methyl or hydrogen. In yet another aspect, R³ is CH₂OH or hydrogen.

In one aspect, R is the group OR⁴ and R⁴ is C₁₋₄ alkyl in the compound of formula (I).

In another aspect, R⁴ is propyl or ethyl.

In yet another aspect, the compound of formula (I) is selected from a 1-methylnicotinamide salt or a 1-methyl-N′-hydroxymethylnicotinamide salt.

In one aspect, the method comprises administering to the subject a compound of formula (I), wherein the compound is selected from a 1-methylnicotinic acid ethyl ester salt or a 1-methylnicotinic acid propyl ester salt. In another aspect, the compound of formula (I) is selected from a 1-methylnicotinic acid salt. In yet another aspect, the compound of formula (I) is selected from 1-methylnicotinamide chloride, 1-methylnicotinamide citrate, 1-methylnicotinamide lactate, 1-methyl-N′-hydroxymethylnicotinamide chloride, 1-methylnicotinic acid chloride, 1-methylnicotinic acid ethyl ester chloride or 1-methylnicotinic acid propyl ester chloride.

In one aspect, the gastrointestinal disorder is associated with the development and progress of gastric mucosal lesion. In another aspect, the gastrointestinal disorder is associated with irritant-, ethanol-, stress-, or ischemia/reperfusion-induced gastric lesions. In yet another aspect, the gastrointestinal disorder is associated with undue gastric acid secretion, peptic ulcer, gastric mucosal damage, stress ulcers, gastric hyperacidity, dyspepsia, gastroparesis, Zollinger-Ellison syndrome, gastroesophageal reflux disease, short-bowel (anastomosis) syndrome, hypersecretory states associated with systemic mastocytosis or basophilic leukemia and hyperhistaminemia, or a bleeding peptic ulcer, with the proviso that the gastrointestinal disorder is not gastric ulcer or duodenal ulcer.

In one aspect, the method comprises administering to the subject a compound of formula (I) orally, nasally, rectally, intravaginally, parenterally, buccally, sublingually, intragastrically or topically. Preferably, the subject is a mammal. More preferably, the subject is a human.

In another aspect, the compound is formulated using one or more pharmaceutically acceptable excipients selected from the group consisting of starch, sugar, cellulose, diluent, granulating agent, lubricant, binder, disintegrating agent, wetting agent, emulsifier, coloring agent, release agent, coating agent, sweetening agent, flavoring agent, perfuming agent, preservative, antioxidant, plasticizer, gelling agent, thickener, hardener, setting agent, suspending agent, surfactant, humectant, carrier, stabilizer, and any combinations thereof.

In one aspect, the method comprises administering to the subject a composition comprising a compound of formula (I) and an anti-ulcer agent. In one aspect, the anti-ulcer agent is selected from the group consisting of an antibacterial agent, an alginate, a prokinetic agent, an H₂-receptor antagonist, a proton pump inhibitor (PPI), a promotility agent, an antacid, sucralfate, heparin, and any combination thereof.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a chart depicting the effects of administering ID-MNA, NA, or vehicle (control) on gastric output in rats having chronic gastric fistula (GF). Administration of 1-MNA suppressed gastric acid output more effectively than NA in the rat model.

FIG. 2 is a chart demonstrating that pretreatment with 1-MNA resulted in decreased lesion number and increased gastric blood flow in WRS-induced gastric lesion animal models. 1-MNA was able to reduce lesion number more effectively than NA.

FIG. 3 is a chart demonstrating the effectiveness of administering 1-MNA in reducing gastric lesions in the present or absence of pretreatment with indomethacin (5 mg/kg i.p.), SC-560 (5 mg/kg i.g.) or rofecoxib (10 mg/kg i.g.). Pretreatment with indomethacin, SC-560, or rofecoxib inhibited the protection offered by 1-MNA against WRS-induced gastric lesions. Administration of 16,16-dimethyl-PGE2 reduced the gastric damage induced by either indomethacin, SC-560, or rofecoxib.

FIG. 4 is a chart demonstrating that administration of 1-MNA decreased the plasma levels of both IL-1β and TNFα in the WRS model. Pretreatment with indomethacin, SC-560, or rofecoxib inhibited the protection by 1-MNA with respect to gastric lesions.

FIG. 5 is a chart demonstrating the effectiveness of 1-MNA on gastric lesions with respect to capsaicin-induced denervation in preconditioned gastric mucosa. Capsaicin denervation was able to inhibit the protection induced by 1-MNA with respect to gastric lesions. The addition of CGRP counteracted the deleterious effects of capsaicin-induced denervation.

FIG. 6 is a chart demonstrating the effectiveness of 1-MNA on gastric mucosa injury in the WRS-induced ulceration model in the presence of capsazepine (a competitive vanilloid receptor antagonist). Capsazepine abolished the protective property of 1-MNA against gastric mucosa injury as measured by mean lesion number and gastric blood flow. The addition of CGRP was able to counteract the deleterious effects of casazepine.

FIG. 7 is a chart demonstrating that 1-MNA had a therapeutic effect in the combined diabetes and stress-induced gastric damage model. 1-MNA was able to reduce mean lesion number and decrease both plasma levels of IL-1β and TNFα in this animal model.

FIG. 8 is a chart demonstrating that pretreatment with 1-MNA resulted in decreased lesion number and increased gastric blood flow in ethanol-induced gastric lesion animal models. 1-MNA was able to reduce lesion number and increase gastric blood flow more effectively than NA in this animal model.

FIG. 9 is a chart demonstrating that the mean lesion number in ethanol-induced gastric lesion animal models pretreated with indomethacin, SC-560, or rofecoxib was significantly reduced in the presence of 1-MNA.

FIG. 10 is a chart demonstrating that pretreatment with 1-MNA resulted in decreased lesion number and increased gastric blood flow in ASA-induced gastric lesion animal models. 1-MNA was able to reduce lesion number and increase gastric blood flow more effectively than NA in this animal model.

FIG. 11 is a chart demonstrating that pretreatment with 1-MNA in the ASA-induced gastric lesion model contributed to lowering the levels of MPO, increasing the levels of SOD, and decreasing the levels of MDA and 4-HNE.

FIG. 12 is a chart demonstrating that 1-MNA was able to reduce mean lesion number, increase luminal NO, and increase GBF effectively as compared to the protective properties of ranitidine (histamine H2-receptor antagonist), NO-ASA (NO-releasing aspirin), and SIN-1 (3-morpholino-sydnonymide; a donor of nitric oxide).

FIG. 13 is a chart demonstrating that pretreatment with 1-MNA resulted in decreased lesion number and increased gastric blood flow in I/R-induced gastric lesion animal models. Administration of L-NNA was observed to reverse the therapeutic effects of 1-MNA on gastric lesions in this animal model.

FIG. 14 is a chart demonstrating that 1-MNA was able to reduce the ulcer area in rats given rofecoxib as well as increase the GBF in the ulcer area. The combination of 1-MNA with DM PGE₂ in rats given rofecoxib served to reduce the ulcer area at a higher level in the rat as well as increase the GBF in the ulcer area as compared to administration of 1-MNA alone.

### DETAILED DESCRIPTION OF THE INVENTION

The disclosure presented herein demonstrates that a pyridinium salt, namely, 1-methyl nicotinamide (1-MNA), is useful for treating gastrointestinal disorders. Gastrointestinal disorders include, but are not limited to, peptic ulcers, stress ulcers, gastric hyperacidity, dyspepsia, gastroparesis, Zollinger-Ellison syndrome, gastroesophageal reflux disease, short-bowel (anastomosis) syndrome, hypersecretory states associated with systemic mastocytosis or basophilic leukemia and hyperhistaminemia, and bleeding peptic ulcers that result, for example, from neurosurgery, head injury, severe body trauma or burns, with the proviso that the gastrointestinal disorder is not gastric ulcer or duodenal ulcer.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

The phrase “gastrointestinal disorder”, as used herein, refers to any disease or disorder of the upper gastrointestinal tract of a patient including, for example, peptic ulcers, stress ulcers, gastric hyperacidity, dyspepsia, gastroparesis, Zollinger-Ellison syndrome, gastroesophageal reflux disease, short-bowel (anastomosis) syndrome, hypersecretory states associated with systemic mastocytosis or basophilic leukemia and hyperhistaminemia, and bleeding peptic ulcers that result, for example, from neurosurgery, head injury, severe body trauma or burns, with the proviso that the gastrointestinal disorder is not gastric ulcer or duodenal ulcer.

“Gastrointestinal disorder” can also refer to a disorder of the gastrointestinal tract, including the small and large intestines and the rectum, and/or symptoms usually attributed to a dysfunction of one or more of these organs, such as diarrhea, constipation and/or abdominal and lower abdominal cramping or pain. It is understood that gastrointestinal disorders include both disorders for which an organic cause (e.g. infection by a parasite) is known and disorders for which no organic cause can be ascertained, such as irritable bowel syndrome (IBS). Gastrointestinal disorders, therefore, include, but are not limited to, irritable bowel syndrome, functional diarrhea, ulcerative colitis, collagenous colitis, microscopic colitis, lymphocytic colitis, inflammatory bowel disease, Crohn's disease, and infectious diarrhea such as diarrhea associated with amebiasis, giardiasis, a viral infection, cytomegalovirus infection, or a pathogenic bacterial infection. The bacterial infection may, for example, be an infection by a bacterium selected from the group consisting of a bacterium of the genus Escherichia, an Escherichia coli 0157:H7 bacterium, a bacterium of the genus Salmonella, a bacterium of the genus Shigella, a bacterium of the genus Campylobacter, a bacterium of the species Campylobacter jejuni, and a bacterium of the genus Yersinia.

“Gastric and intestinal protection” in this connection is understood as meaning the prevention and treatment of gastrointestinal diseases, in particular of gastrointestinal inflammatory diseases and lesions (such as, for example, gastritis, hyperacidic or medicament-related functional dyspepsia), which can be caused, for example, by microorganisms (e.g. Helicobacter pylori), bacterial toxins, medicaments (e.g. certain anti-inflammatories and antirheumatics), chemicals (e.g. ethanol), gastric acid or stress situations.

“Upper gastrointestinal tracf” refers to the esophagus, the stomach, the duodenum and the jejunum.

“Ulcers” refers to lesions of the upper gastrointestinal tract lining that are characterized by loss of tissue. Such ulcers include esophageal ulcer, Meckel's Diverticulum ulcer and gastritis.

“Patient” refers to animals, preferably mammals, more preferably humans.

The term “treatment” or “treating,” as used herein, is defined as the application or administration of a therapeutic agent, i.e., a compound of the invention (alone or in combination with another pharmaceutical agent), to a subject, or application or administration of a therapeutic agent to an isolated tissue or cell line from a subject (e.g., for diagnosis or ex vivo applications), who has a gastrointestinal disorder, a symptom of a gastrointestinal disorder or a predisposition toward a gastrointestinal disorder, with the purpose to cure, heal, alleviate, relieve, alter, remedy, ameliorate, improve or affect the gastrointestinal disorder, the symptoms of the gastrointestinal disorder or the gastrointestinal disorder. Such treatments may be specifically tailored or modified, based on knowledge obtained from the field of pharmacogenomics.

The term “subject” includes living organisms in which gastrointestinal disorders can occur, or that are susceptible to gastrointestinal disorders. The term “subject” includes animals (e.g., mammals, e.g., cats, dogs, horses, pigs, cows, goats, sheep, rodents, e.g., mice or rats, rabbits, squirrels, bears, primates e.g., chimpanzees, monkeys, gorillas, and humans), as well as chickens, ducks, geese, and transgenic species thereof; and cells, e.g., immortalized or nonimmortalized cells, derived therefrom.

As used herein, a “therapeutically effective amount” is the amount of material sufficient to provide a beneficial effect to the subject to which the materials are administered.

The term “pharmaceutically acceptable,” as used herein, refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula (I) and a co-agent, are both administered to a patient simultaneously in the form of a single entity or dosage. The term “non-fixed combination” means that the active ingredients, e.g. a compound of formula (I) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound of formula (I) with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “prevent” or “prevention,” in relation to a gastrointestinal disorder or disease, means no gastrointestinal disorder or disease development if none had occurred, or no further gastrointestinal disorder or disease development if there had already been development of the gastrointestinal disorder or disease. Also considered is the ability of one to prevent some or all of the symptoms associated with the gastrointestinal disorder or disease.

### DESCRIPTION

The present invention includes a method of preventing and/or reducing gastrointestinal injury, wherein said gastrointestinal injury is not gastric ulcer or duodenal ulcer. The method involves administering a compound of formula (I), preferably the compound is 1-MNA, to a subject who has suffered, is at risk to suffer, or to prevent or reduce gastrointestinal injury, wherein said gastrointestinal injury is not gastric ulcer or duodenal ulcer.

The invention is based partly on the discovery that pretreatment with 1-MNA exhibited therapeutic benefits in animal models of induced gastric lesions. Pretreatment with 1-MNA modulated gastric blood flow and gastric tissue activities of MDA and SOD, as well as pro-inflammatory cytokines IL-1β and TNF-α.

### COMPOUNDS OF THE INVENTION

The nicotinamide derivatives of the invention can be synthesized using techniques well-known in the art of organic synthesis.

In one aspect, the nicotinamide derivatives of the instant invention are represented by the formula I:

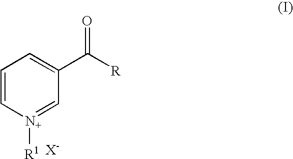

(or pharmaceutically acceptable salts thereof) wherein R is the group NR²R³ or the group OR⁴; R¹ is methyl; R¹ and R⁴ each independently is hydrogen or C₁₋₄alkyl; R³ is hydrogen, C₁₋₄alkyl or CH₂OH; and X is a physiologically suitable counter-anion. In one embodiment, R is the group NR²R³. In another embodiment, R² is methyl or hydrogen. In another embodiment, R³ is CH₂OH or hydrogen. In yet another embodiment, R is the group OR⁴, and R⁴ is C₁₋₄ alkyl. In another embodiment, R⁴ is propyl or ethyl.

In a preferred embodiment, the compound of formula (I) is selected from a 1-methylnicotinamide salt or a 1-methyl-N′-hydroxymethylnicotinamide salt. In another preferred embodiment, the compound of formula (I) is selected from a 1-methylnicotinic acid ethyl ester salt or a 1-methylnicotinic acid propyl ester salt. In another preferred embodiment, formula (I) is selected from a 1-methylnicotinic acid salt. In another preferred embodiment, the salt of formula (I) is a chloride, benzoate, salicylate, acetate, citrate or lactate.

In another preferred embodiment, the compound of formula (I) is selected from 1-methylnicotinamide chloride, 1-methylnicotinamide citrate, 1-methylnicotinamide lactate, 1-methyl-N′-hydroxymethylnicotinamide chloride, 1-methylnicotinic acid chloride, 1-methylnicotinic acid ethyl ester chloride or 1-methylnicotinic acid propyl ester chloride.

Without being bound by theory, it is believed that the compounds of the invention are effective in treating gastrointestinal disorders for the following reasons: on the surface of the endothelium, polyanionic molecules, as glycosaminoglycans, are present, and it would be expected that the molecules able to manifest some endothelial potential should be bound to the endothelium. The nicotinamide compounds of formula (I), which are positively charged, bind to the negatively charged glycosamionoglycans present on the endothelium surface due to electrostatic interactions. This binding can result in manifestation of various endothelial effects, some of which can be positive from pharmacologic view point, for example release of NO and/or prostacyclin. Further, this activity can result in the treatment or prevention of gastrointestinal disorders.

As used herein, the language “pharmaceutically acceptable salt” refers to a salt of the administered compounds prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like.

The term “alkyl” refers to saturated aliphatic groups, including straight-chain alkyl groups, branched-chain alkyl groups, cycloalkyl, heterocyclyl, cycloalkyl (alicyclic) groups, alkyl substituted cycloalkyl groups, and cycloalkyl substituted alkyl groups. In preferred embodiments, a straight chain or branched chain alkyl has 30 or fewer carbon atoms in its backbone (e.g., C₁-C₃₀ for straight chain, C₃-C₃₀ for branched chain), and more preferably has 20 or fewer carbon atoms in the backbone. Likewise, preferred cycloalkyls have from 3-10 carbon atoms in their ring structure, and more preferably have, 3-8 carbon atoms in their ring structure and even more preferably have 5, 6 or 7 carbons in their ring structure.

Moreover, alkyl (e.g., methyl, ethyl, propyl, butyl, pentyl, hexyl, etc.) include both “unsubstituted alkyl” and “substituted alkyl”, the latter of which refers to alkyl moieties having substituents replacing a hydrogen atom on one or more carbon atoms of the hydrocarbon backbone, which allow the molecule to perform its intended function.

Examples of substituents of the invention, which are not intended to be limiting, include moieties selected from straight or branched alkyl (preferably C₁-C₈), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃ (CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₄R′ (e.g., —SO₃H, —OSO₃H—), (CR′R″)₀₋₃₀(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃ (C₃₈cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, oxime, sulfhydryl, alkylthio, arylthio, thiocarboxylate, sulfate, sulfonato, sulfamoyl, sulfonamido, nitro, trifluoromethyl, cyano, azido, heterocyclyl, or an aromatic or heteroaromatic moiety. In certain embodiments, a carbonyl moiety (C═O) may be further derivatized with an oxime moiety, e.g., an aldehyde moiety may be derivatized as its oxime (—C═N—OH) analog. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

### Combination Therapies

The nicotinamide derivatives of the present invention are intended to be useful, e.g., in the methods of present invention, in combination with one or more additional compounds useful for treating a gastrointestinal disorder. These additional compounds may comprise compounds of the present invention or compounds, e.g., commercially available compounds, known to treat, prevent, or reduce the symptoms of a gastrointestinal disorder.

The phrase “combination therapy” means the administration of a composition of the present invention in conjunction with another pharmaceutical agent. The therapeutic compounds that make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Substantially simultaneous administration can be accomplished, for example, by administering to the subject a single tablet or capsule having a fixed ratio of each therapeutic agent or in multiple, single capsules, or tablets for each of the therapeutic agents. Sequential or substantially simultaneous administration of each therapeutic agent can be effected by any appropriate route. Thus, a regimen may call for sequential administration of the therapeutic compounds with spaced-apart administration of the separate, active agents. The time period between the multiple administration steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval.

The therapeutic compounds of the combined therapy, whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, by inhalation spray, rectally, topically, buccally (for example, sublingual), or parenterally (for example, subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components.

Combination therapy includes, for example, administration of a composition of the present invention in conjunction with another pharmaceutical agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually substantially simultaneously, minutes, hours, days, weeks, months or years depending upon the combination selected).

A combination of compounds described herein can either result in synergistic increase in effectiveness against a gastrointestinal disorder, relative to effectiveness following administration of each compound when used alone, or such an increase can be additive. Compositions described herein typically include lower dosages of each compound in a composition, thereby avoiding adverse interactions between compounds and/or harmful side effects. Furthermore, normal amounts of each compound when given in combination could provide for greater efficacy in subjects who are either unresponsive or minimally responsive to each compound when used alone.

A synergistic effect can be calculated, for example, using suitable methods such as, for example, the Sigmoid-Emax equation (Holford and Scheiner, 1981 Clin. Pharmacokinet. 6: 429-453), the equation of Loewe additivity (Loewe and Muischnek, 1926 Arch. Exp. Pathol. Pharmacol. 114: 313-326) and the median-effect equation (Chou and Talalay, 1984 Adv. Enzyme Regul. 22: 27-55). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

The present methods, kits, and compositions can be used in combination with another pharmaceutical agent that is indicated for treating or preventing a gastrointestinal disorder, such as, for example, an antibacterial agent, an alginate, a prokinetic agent, a H₂-antagonist (also known as H2-receptor antagonists or histamine H₂ receptor blockers), a proton pump inhibitor (PPI), a promotility agent, an antacid, or sucralfate, which are commonly administered to minimize the pain and/or complications related to this disorder.

H₂-receptor antagonists inhibit the action of histamine on the parietal cell, inhibiting acid secretion. Examples of H₂-receptor antagonists include cimetidine, nizatidine, ranitidine hydrochloride, lansoprazole, and rabeprazole. Cimitidine inhibits histamine at H₂-receptors of the gastric parietal cells, resulting in reduced gastric acid secretion, gastric volume, and reduced hydrogen ion concentrations. Nizatidine competitively inhibits histamine at H₂-receptors of gastric parietal cells, also resulting reduced gastric acid secretion, gastric volume, and reduced hydrogen ion concentrations. Lansoprazole decreases gastric acid secretion by inhibiting the parietal cell H⁺/K⁺ ATP pump, and is used to relieve symptoms of active duodenal ulcers and erosive esophagitis. Rabeprazole also decreases gastric acid secretion by inhibiting the parietal cell H⁺/K⁺ ATP pump, and is used for short-term treatment and symptomatic relief of gastritis, and for the treatment of active duodenal ulcers, and all grades of erosive esophagitis.

Proton pump inhibitors (PPI) are potent inhibitors of gastric acid secretion, inhibiting H⁺/K⁺-ATPase, the enzyme involved in the final step of hydrogen ion production in the parietal cells. The term “proton pump inhibitor” includes, but is not limited to, omeprazole, lansoprazole, rabeprazole, pantoprazole and leminoprazole, including isomers, enantiomers and tautomers thereof, and alkaline salts thereof. Proton pump inhibitors typically include benzimidazole compounds. The following patents describe various benzimidazole compounds suitable for use in the invention described herein: U.S. Pat. No. 4,045,563, U.S. Pat. No. 4,255,431, U.S. Pat. No. 4,359,465, U.S. Pat. No. 4,472,409, U.S. Pat. No. 4,508,905, JP-A-59181277, U.S. Pat. No. 4,628,098, U.S. Pat. No. 4,738,975, U.S. Pat. No. 5,045,321, U.S. Pat. No. 4,786,505, U.S. Pat. No. 4,853,230, U.S. Pat. No. 5,045,552, and U.S. Pat. No. 5,312,824. All of the above patents are hereby incorporated by reference. Proton pump inhibitors, e.g. omeprazole and its pharmaceutically acceptable salts, which are used in accordance with the invention, are known compounds and can be produced by known processes. In certain preferred embodiments, the proton pump inhibitor is omeprazole, either in racemic mixture or only the (−)-enantiomer of omeprazole (i.e. esomeprazole), as set forth in U.S. Pat. No. 5,877,192, hereby incorporated by reference. Accordingly, the present invention includes pharmaceutical formulations combining a proton pump inhibitor such as omeprazole with 1-MNA.

Other agents include a nitric oxide donor, bismuth-containing reagents and an anti-viral. These drugs have certain disadvantages associated with their use. Some of these drugs are not completely effective in the treatment of the aforementioned conditions and/or produce adverse side effects, such as mental confusion, constipation, diarrhea, and thrombocytopenia. H₂-antagonists, such as ranitidine and cimetidine, are relatively costly modes of therapy, particularly in NPO patients (a Latin abbreviation for “nothing by mouth”), which frequently require the use of automated infusion pumps for continuous intravenous infusion of the drug. However, when used in conjunction with the present invention, that is, in combination therapy, many if not all of these unwanted side effects can be reduced or eliminated. The reduced side effect profile of these drugs is generally attributed to, for example, the reduced dosage necessary to achieve a therapeutic effect with the administered combination.

In another example, the present methods, kits, and compositions can be used in combination with other pharmaceutical agents, including but not limited to: sleep aids including but not limited to a benzodiazepine hypnotic, non-benzodiazepine hypnotic, antihistamine hypnotic, antidepressant hypnotic, herbal extract, barbiturate, peptide hypnotic, triazolam, brotizolam, loprazolam, lormetazepam, flunitrazepam, flurazepam, nitrazepam, quazepam, estazolam, temazepam, lorazepam, oxazepam, diazepam, halazepam, prazepam, alprazolam, chlordiazepoxide, clorazepate, an imidazopyridine or pyrazolopyrimidine hypnotic, zolpidem or zolpidem tartarate, zopiclone, eszopiclone, zaleplon, indiplone, diphenhydramine, doxylamine, phenyltoloxamine, pyrilamine, doxepin, amtriptyline, trimipramine, trazodon, nefazodone, buproprion, bupramityiptyline, an herbal extract such as valerian extract or amentoflavone, a hormone such as melatonin, or gabapeptin; motility agents, including but not limited to 5-HT inhibitors such as cisapride, domperidone, and metoclopramide, and agents useful for treating irritable bowel syndrome.

### Formulations for Administration

In another embodiment, the present invention is directed to a packaged pharmaceutical composition comprising a container holding a therapeutically effective amount of a nicotinamide derivative of formula I, alone and in combination with a second pharmaceutical agent; and instructions for using the compound to treat, prevent, or reduce one or more symptoms of one or more gastrointestinal disorder in a subject.

In one aspect, the invention includes a combined formulation comprising a therapeutically effective amount of a nicotinamide derivative of formula I and an anti-ulcer agent including, but not limited to an antibacterial agent, an alginate, a prokinetic agent, an H₂-receptor antagonist, a proton pump inhibitor (PPI), a promotility agent, an antacid, sucralfate, heparin, and any combination thereof.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation can then be provided to a tablet press or be encapsulated for preparing the oral dosage form. Melt granulation improves the dissolution rate and bioavailability of an active (i.e. drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

In a further embodiment, the present invention relates to a pharmaceutical oral dosage form taking the form of a multi-layer tablet. The multi-layer tablet includes a layer comprising one or more nicotinamide derivative of formula I, which layer is prepared by the above-described melt granulation procedure. The multi-layer tablet further includes a layer comprising one or more anti-ulcer agent such as one or more proton pump inhibitors and/or one or more H₂-receptor antagonist. The tablet may optionally further include a separating layer between the layers containing the active ingredients. In a particular embodiment, the pharmaceutical oral dosage form is a bi-layer tablet comprising misoprostol or physiologically acceptable salts thereof.

In a further embodiment, the present invention relates to a method for manufacturing a multi-layer tablet comprising a layer providing for the delayed release of one or more nicotinamide derivative of formula I, and a further layer providing for the immediate release of an anti-ulcer agent such as a proton pump inhibitor and/or an H₂-receptor antagonist. Using a wax/pH-sensitive polymer mix, a gastric insoluble composition can be obtained in which the active ingredient is entrapped, ensuring its delayed release.

Formulations can be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., other analgesic agents. For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing one or more gastrointestinal disorder in a subject.

Another embodiment of the invention is a pharmaceutical composition comprising a therapeutically effective amount of a nicotinamide compound of formula I and a pharmaceutically acceptable carrier.

The language “therapeutically effective amount” describes the amount of nicotinamide derivative of formula I of the invention that is effective to treat one or more gastrointestinal disorders in a subject.

The language “pharmaceutically acceptable carrier” includes a pharmaceutically acceptable material, composition or carrier, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent that delays absorption, for example, aluminum monostearate or gelatin. In one embodiment, the pharmaceutically acceptable carrier is not DMSO alone.

The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Administration of the compositions of the present invention to a subject to be treated can be carried out using known procedures, at dosages and for periods of time effective to inhibit gastrointestinal disorders in the subject. An effective amount of the therapeutic compound necessary to achieve a therapeutic effect may vary according to factors such as the state of the disease or disorder in the subject, the age, sex, and weight of the subject, and the ability of the therapeutic compound to inhibit the gastrointestinal disorders in the subject. Dosage regimens can be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention (e.g., methyl nicotinamide) is between 1 and 500 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound of the present invention employed, the time of administration, the rate of excretion of the particular compound being employed, the duration of the treatment, other drugs, compounds or materials used in combination with the particular compound employed, the age, sex, weight, condition, general health and prior medical history of the patient being treated, and like factors well, known in the medical arts.

A medical doctor, e.g., physician or veterinarian, having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The regimen of administration can affect what constitutes an effective amount. The therapeutic formulations can be administered to the subject either prior to or after the onset of a gastrointestinal disorder. Further, several divided dosages, as well as staggered dosages, can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

In particular embodiments, it is especially advantageous to formulate compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a gastrointestinal disorder in subjects.

### Oral Administration

For oral administration, the compounds of the invention can be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

### Transmucosal Administration

Transmucosal administration is carried out using any type of formulation or dosage unit suitable for application to mucosal tissue. For example, the selected active agent can be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

### Transurethal Administration

With regard to transurethal administration, the formulation can comprise a urethral dosage form containing the active agent and one or more selected carriers or excipients, such as water, silicone, waxes, petroleum jelly, polyethylene glycol (“PEG”), propylene glycol, liposomes, sugars such as mannitol and lactose, and/or a variety of other materials. A transurethral permeation enhancer can be included in the dosage from. Examples of suitable permeation enhancers include dimethylsulfoxide (“DMSO”), dimethyl formamide (“DMF”), N,N-dimethylacetamide (“DMA”), decylmethylsulfoxide

(“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone™ from Nelson Research & Development Co., Irvine, Calif.), SEPAL (available from Macrochem Co., Lexington, Mass.), surfactants as discussed above, including, for example, Tergitol™, Nonoxynol-9™ and TWEEN-80™, and lower alkanols such as ethanol.

### Transrectal Administration

Transrectal dosage forms may include rectal suppositories, creams, ointments, and liquid formulations (enemas). The suppository, cream, ointment or liquid formulation for transrectal delivery comprises a therapeutically effective amount of the selected active agent and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration may be in the range of from about 10 minutes to about 6 hours, e.g., less than about 3 hours.

### Vaginal or Perivaginal Administration

Vaginal or perivaginal dosage forms may include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit can be fabricated to disintegrate rapidly or over a period of several hours. The time period for complete disintegration may be in the range of from about 10 minutes to about 6 hours, e.g., less than about 3 hours.

### Intranasal or Inhalation Administration

The active agents may also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators can be used. For liquid formulations, the active agent can be formulated into a solution, e.g., water or isotonic saline, buffered or unbuffered, or as a suspension. In certain embodiments, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers can also include nasal gels, creams, pastes or ointments with a viscosity of, e.g., from about 10 to about 6500 cps, or greater, depending on the desired sustained contact with the nasal mucosal surfaces. Such carrier viscous formulations may be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation can take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations can contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation can also comprise dry powder formulations, particularly insufflations in which the powder has an average particle size of from about 0.1 μm to about 50 gm, e.g., from about 1 μm to about 25 μm.

### Topical Formulations

Topical formulations can be in any form suitable for application to the body surface, and may comprise, for example, an ointment, cream, gel, lotion, solution, paste or the like, and/or may be prepared so as to contain liposomes, micelles, and/or microspheres. In certain embodiments, topical formulations herein are ointments, creams and gels.

### Transdermal Administration

Transdermal compound administration, which is known to one skilled in the art, involves the delivery of pharmaceutical compounds via percutaneous passage of the compound into the systemic circulation of the patient. Topical administration can also involve the use of transdermal administration such as transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions can include creams, sprays, lotions, gels, ointments, eye drops, nose drops, ear drops, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application. The compositions can also be applied topically using a transdermal system, such as one of an acrylic-based polymer adhesive with a resinous crosslinking agent impregnated with the composition and laminated to an impermeable backing.

Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir that, in this case, may be either a polymeric matrix as described above, or it may be a liquid or hydrogel reservoir, or may take some other form.

### Intrathecal Administration

One common system utilized for intrathecal administration is the APT Intrathecal treatment system available from Medtronic, Inc. APT Intrathecal uses a small pump that is surgically placed under the skin of the abdomen to deliver medication directly into the inirathecal space. The medication is delivered through a small tube called a catheter that is also surgically placed. The medication can then be administered directly to cells in the spinal cord involved in conveying sensory and motor signals associated with lower urinary tract disorders.

### Intravesical Administration

The term intravesical administration is used herein in its conventional sense to mean delivery of a drug directly into the bladder. Suitable methods for intravesical administration can be found, for example, in U.S. Pat. Nos. 6,207,180 and 6,039,967.

### Additional Administration Forms

Additional dosage forms of this invention include dosage forms as described in U.S. Pat. No. 6,340,475, U.S. Pat. No. 6,488,962, U.S. Pat. No. 6,451,808, U.S. Pat. No. 5,972,389, U.S. Pat. No. 5,582,837, and U.S. Pat. No. 5,007,790. Additional dosage forms of this invention also include dosage forms as described in U.S. patent application Ser. No. 20030147952, U.S. patent application Ser. No. 20030104062, U.S. patent application Ser. No. 20030104053, U.S. patent application Ser. No. 20030044466, U.S. patent Application Ser. No. 20030039688, and U.S. patent application Ser. No. 20020051820. Additional dosage forms of this invention also include dosage forms as described in WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present invention can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release that is longer that the same amount of agent administered in bolus form.

For sustained release, the compounds can be formulated with a suitable polymer or hydrophobic material that provides sustained release properties to the compounds. As such, the compounds for use the method of the invention can be administered in the form of microparticles for example, by injection or in the form of wafers or discs by implantation.

In a preferred embodiment of the invention, the nicotinamide compounds of formula I are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any and all whole or partial increments there between after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments there between after drug administration.

### Dosing

The therapeutically effective amount or dose of a compound of the present invention will depend on the age, sex and weight of the patient, the current medical condition of the patient and the nature of the gastrointestinal disorder being treated. The skilled artisan will be able to determine appropriate dosages depending on these and other factors.

A suitable dose of a compound of the present invention can be in the range of from about 0.001 mg to about 500 mg per day, such as from about 0.01 mg to about 100 mg, for example, from about 0.05 mg to about 50 mg, such as about 0.5 mg to about 25 mg per day. The dose can be administered in a single dosage or in multiple dosages, for example from 1 to 4 or more times per day. When multiple dosages are used, the amount of each dosage can be the same or different. For example a dose of 1 mg per day can be administered as two 0.5 mg doses, with about a 12 hour interval between doses.

Nicotinamide derivatives of the invention for administration can be in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, and any and all whole or partial increments there between.

In some embodiments, a dose of a nicotinamide derivative of the invention is between about 0.0001 mg and about 25 mg. In some embodiments, a dose of a nicotinamide derivative of the invention used in compositions described herein is less than about 100 mg, or less than about 80 mg, or less than about 60 mg, or less than about 50 mg, or less than about 30 mg, or less than about 20 mg, or less than about 10 mg, or less than about 5 mg, or less than about 2 mg, or less than about 0.5 mg. Similarly, in some embodiments, a dose of a second compound as described herein is less than about 1000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments there between.

It is understood that the amount of compound dosed per day can be administered every day, every other day, every 2 days, every 3 days, every 4 days, every 5 days, etc. For example, with every other day administration, a 5 mg per day dose can be initiated on Monday with a first subsequent 5 mg per day dose administered on Wednesday, a second subsequent 5 mg per day dose administered on Friday, etc.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including reaction times, reaction size/volume, and experimental reagents, such as solvents, catalysts, pressures, atmospheric conditions, e.g., nitrogen atmosphere, and reducing/oxidizing agents, etc., with art-recognized alternatives and using no more than routine experimentation, are within the scope of the present application.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

A variety of factors produce damage to the gastric mucosa, including: systemic events such as thermal stress, or local mucosal application of various irritants. The exposure of gastric mucosa to damaging factors, such as ethanol, water restraint stress, irritants, or ischemia followed by reperfusion, produces pathological changes: inflammatory process, hemorrhagic erosions, even acute ulcers. The basis of these changes is a disturbance of protective mechanisms and disrupture of gastric mucosal barrier. The results presented herein demonstrate the therapeutic benefits of 1-MNA on gastric mucosal damage.

The methods used in experimental induction of gastric lesions in animals include: intragastrical ethanol administration, intragastric administration of aspirin, water immersion restraint stress, as well as ischemia followed by reperfusion. Results from these experiments demonstrate that production of gastric mucosal lesions lead to decrease in gastric blood flow, inflammatory changes expressed by increase IL-1β and TNF-α levels, as well as increase mean lesion number.

### Example 1

### Production of Gastric Lesions

Male Wistar rats, weighing 180-220 grams were fasted for 24 hours before subjecting to experimentation were employed in all studies. The animals were divided into groups for the following types of induced gastric lesions: ethanol-; aspirin (acetylsalicylic acid; ASA)-; stress-; and ischemia-reperfusion (I/R)-induced gastric lesions. In the first group, gastric lesions were produced by intragastric (i.g.) application of ethanol (75%), in the second group by i.g. application of acidified ASA (125 mg/kg), in the third group by animal exposure to 3.5 hours of water immersion (and restraint stress (WRS), in the fourth group by ischemia (by clamping celiac artery for 30 minutes followed by release of the clamp (reperfusion for 3 hours) (I/R).

The following experiments were designed to assess the therapeutic effect of 1-MNA on induced gastric lesions. Vehicle (saline) or 1-MNA (6.25-200 mg/kg i.p.) was administered 30 minutes prior to subjecting the animals to ethanol, ASA, WRS or I/R. In some experiments, vehicle or 1-MNA was administered to the animals with or without pretreatment with the following:
indomethacin (5 mg/kg i.p.), SC-560 (5 mg/kg i.g.) or rofecoxib (10 mg/kg i.g.), (non-selective (indomethacin) and selective COX-1 (SC-560) and COX-2 (rofecoxib) inhibitors);
L-NNA (20 mg/kg i.p.), to suppress the NO-synthase;
capsaicin (125 mg/kg s.c.), to induce the functional ablation of the sensory afferent nerves;
CGRP8-37 (100 μg/kg i.p.) to antagonize CGRP receptors and capsazepine (5 mg/kg s.c.) to inhibit vanilloid (TVRP1) receptors, respectively.

### Example 2

### Measurements of Gastric Lesions

Gastric Blood Flow:

To assess gastric blood flow (GBF) laser Doppler flowmeter (Laserflo, model BPM 403A, Blood Perfusion Monitor, Vasamedics, St. Paul, Minn., USA) was used. The animals were anaesthetized with Vetbutal 50 mg/kg (Biowet, Pu³awy, Poland), then the abdomen was opened and the stomach was exposed to assess the GBF. Blood flow was measured on anterior and posterior wall of stomach. The mean values of these measurements were calculated and expressed as percent change from value recorded in the intact mucosa.

Area of Lesions:

To establish the number and area of gastric lesions, computerized planimetry (Morphomat, Carl Zeiss, Berlin, Germany) was used. Results are expressed as mm.

Determination of Plasma IL-1β and TNFα Levels

A venous blood sample was withdrawn from the vena cava into EDTA-containing vials in order to determine the plasma level of interleukin-1 beta (IL-1β) and tumor necrosis factor alpha (TNFα) by ELISA technique (BioSource International, Camarillo, Calif., USA).

Measurement of Lipid Peroxydation

For lipid peroxydation in investigated groups, the determination of the levels of malondialdehyde (MDA) and 4 hydroxynonenal (4-HNE) was carried out and their levels were used as indicators of lipid peroxidation. The procedure of MDA and 4-HNE determination was following: 600 mg of gastric mucosa was excised. Then 20 ml 0.5 M BHT (butylated hydroxytoluene) was added in order to prevent sample oxidation. This sample was subsequently homogenized in 20 mM Tris for 15 sec. in pH 7.4. The homogenate was centrifuged (3,000 g at 4° C. for 10 minutes). Obtained clear supernatant was stored at −80° C. prior to testing. The colorimetric assay for lipid peroxidation (Bioxytech LPO-586, Oxis, Portland, USA) was used to determine the MDA and 4-HNE tissue concentration. This assay is based on the reaction of a chromogenic reagent N-methyl-2-phenylindole with MDA and 4-HNE at 45° C. This reaction yields a stable chromophore with maximal absorbance at 586 nm. This absorbance was measured by spectrophotometer Marcel s300, Warsaw, Poland. Results were expressed as nanomole per gram of tissue (nmol/g).

Determination of SOD Activity

To determine activity of superoxide dismutase (SOD), a sample of gastric mucosa was taken, as described above. The calorimetric assay for assessment of SOD activity (Bioxytech, SOD-525, Oxis, Portland, USA) was used. This method is based on the SOD-mediated increase in the rate of autooxidation of tetrahydrobenzofluorene in aqueous alkaline solution to yield a chromophore with maximum absorbance at 525 nm. This absorbance was measured by spectrophotometer Marcel s300, Warsaw, Poland. Outcomes were expressed as units per gram of tissue (U/g).

Statistical Analysis

Results are expressed as means±SEM. Statistical analysis was done using nonparametric Mann-Whitney test. Differences with p<0.05 were considered as significant.

### Example 3

### Evaluation of 1-MNA on Gastric Lesions

### Water Immersion and Restraint Stress Model (WRS)

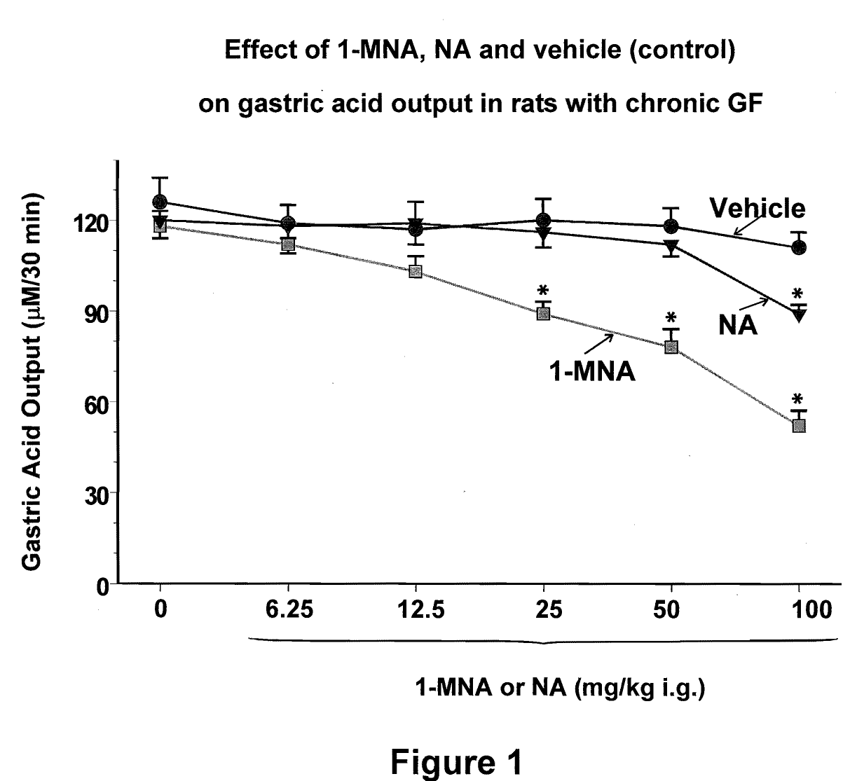

The following experiments were designed to compare the effects of pretreatment with vehicle and 1-MNA or NA on stress-induced gastric lesions and the accompanying changes in gastric blood flow (GBF) and gastric tissue activities of MDA and SOD and plasma pro-inflammatory cytokine IL-1β and TNF-α levels. These sets of experiments were based on the observation that 1-MNA was able to reduce the gastric output in rats having chronic gastric fistula (GF). Rats with chronic GF were administered with 1-MNA, NA, or a vehicle control. It was observed that 1-MNA was able to decrease the gastric acid output in chronic GF rats. The ability of 1-MNA to inhibit gastric output increased with increasing dosage of 1-MNA administered to the rats. 1-MNA was observed to suppress gastric acid output more effectively than NA (FIG. 1).

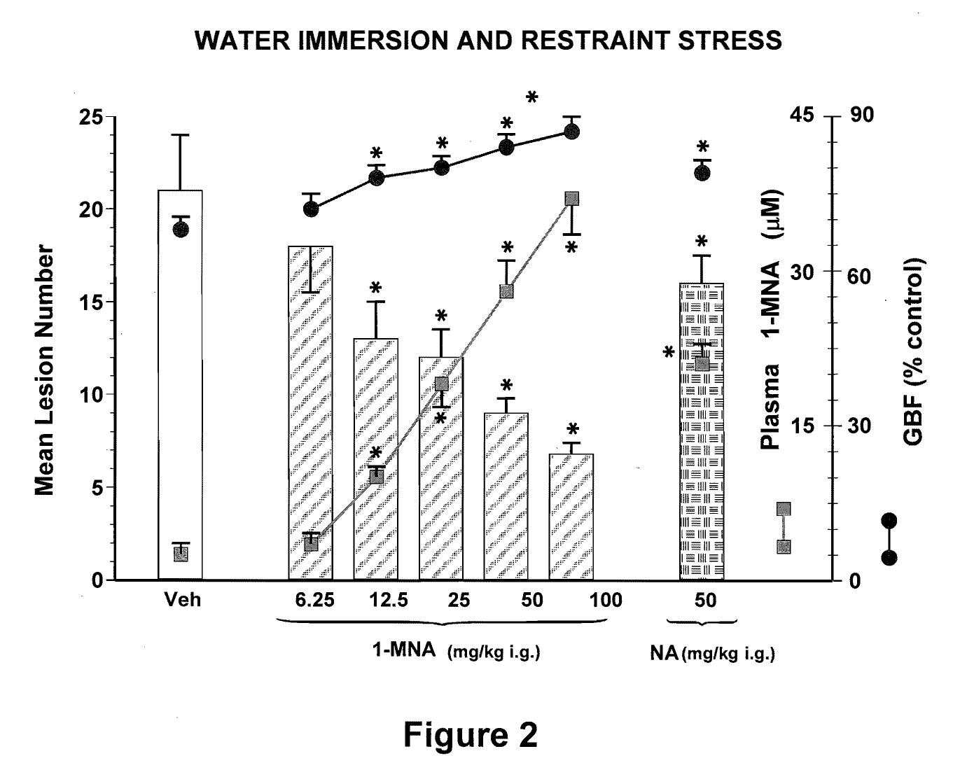

Pretreatment with 1-MNA resulted in decreased lesion number and increased gastric blood flow in WRS-induced gastric lesion animal models (FIG. 2). A comparison of the therapeutic effects of 1-MNA versus NA in the WRS model demonstrated that 1-MNA was able to reduce lesion number more effectively than NA.

Pretreatment with prostaglandin (PG) has been known to prevent the injury of gastric mucosa induced by necrotizing agents. Other mediators such as growth factors, nitric oxide (NO) or calcitonin gene related peptide (CGRP) as well as some gut hormones including gastrin and cholecystokinin (CCK), leptin, ghrelin and gastrin-releasing peptide (GRP) have been also found to protect gastric mucosa against the damage induced by corrosive substances. This protective action of gut hormones has been attributed to the release of PG or activation of sensory nerves because it could be abolished by the pretreatment with indomethacin or large neurotoxic dose of capsaicin and restored by the addition of exogenous prostaglandin E2 (PGE2) or CGRP, respectively.

The following experiments were designed to determine the involvement of gastric acid secretion and potent mediators such as PG, NO, and sensory nerves releasing CGRP in gastroprotective activity of 1-MNA against gastric damage.

### Cox Inhibitors

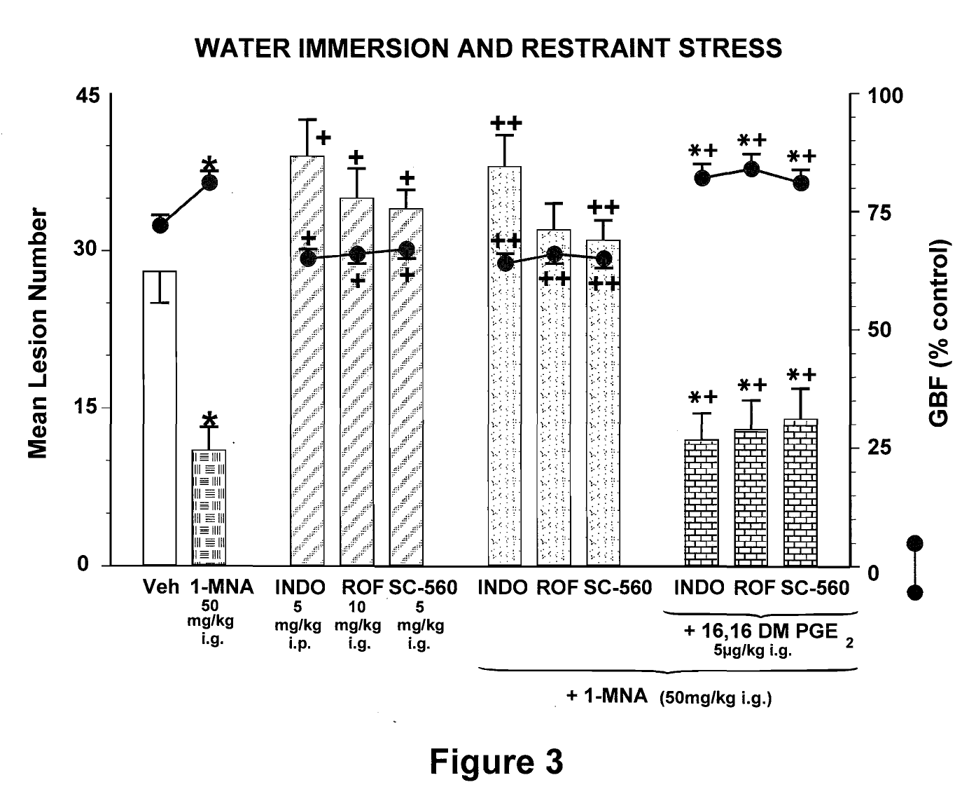

The therapeutic effect of 1-MNA in reducing gastric lesions was further assessed by administering vehicle, NA, or 1-MNA to animals with or without pretreatment with indomethacin (5 mg/kg i.p.), SC-560 (5 mg/kg i.g.) or rofecoxib (10 mg/kg i.g.). Pretreatment with either indomethacin, SC-560, or rofecoxib before the combination of administration of 1-MNA and subjecting the animals to water immersion and restraint stress resulted in the increase of lesion numbers above the values obtained in the absence of either indomethacin, SC-560, or rofecoxib. It was observed that the inhibition by either indomethacin, SC-560, or rofecoxib on the protective property of 1-MNA against WRS-induced gastric lesions was significant (FIG. 3). Administration of 16,16-dimethyl PGE2 was sufficient to decrease the mean lesion number. The administration of 16,16-dimethyl PGE2 was successful in preventing the gastric damage induced by either indomethacin, SC-560, or rofecoxib (FIG. 3). Indomethacin, SC-560, or rofecoxib significantly inhibited the protection by 1-MNA with respect to gastric lesions. Without wishing to be bound by any particular theory, it is believed that 1-MNA protects the gastric mucosa against injury induced by stress, and that the gastroprotective effect of 1-MNA is related to a stimulation of endogenous PGE₂ and PGI₂.

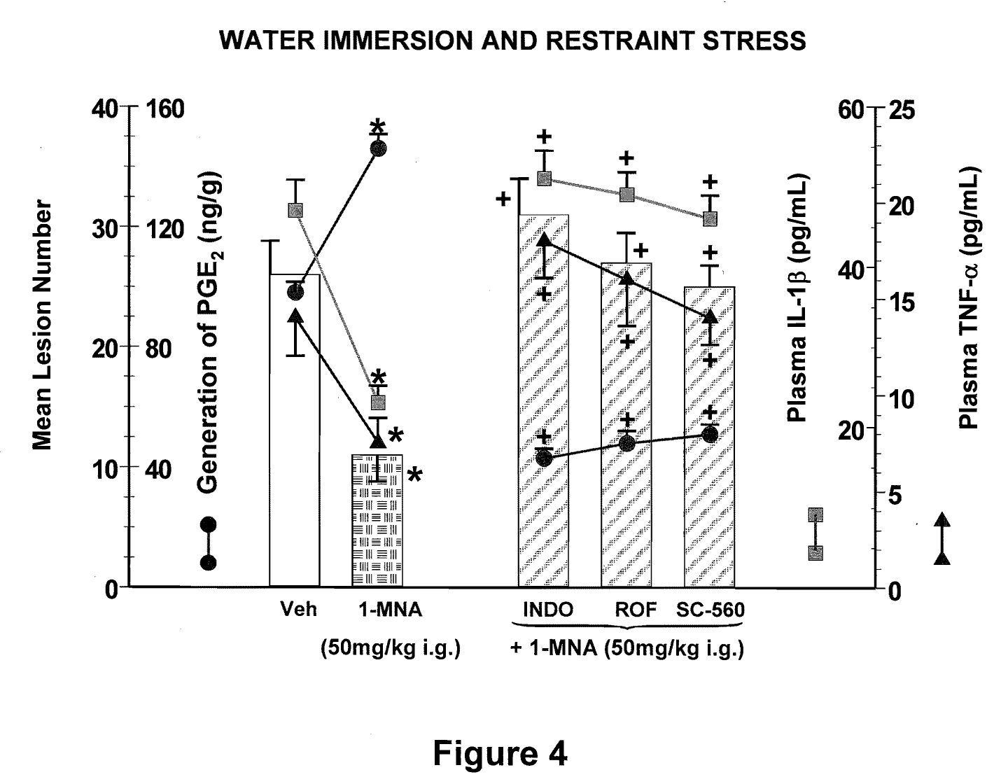

A variety of factors produce damage of gastric mucosa. Gastric lesions are accompanied by a significant increase of proinflammatory cytokines including IL-1β and TNFα plasma levels. Therefore, the next set of experiments was designed to assess the levels of these cytokines as a measurement of prognosis of gastric lesions in the presence and absence of 1-NIA in the WRS model. Consistent with the discovery that 1-MNA was able to decrease the mean lesion number, 1-MNA was also able to decrease the plasma levels of both IL-1β and TNFα. However, pretreatment with indomethacin, SC-560, or rofecoxib significantly inhibited the protective property of 1-MNA on reducing gastric lesions as measured by mean lesion number and plasma levels of IL-1β and TNFα (FIG. 4).

### Capsaicin-Induced Denervation

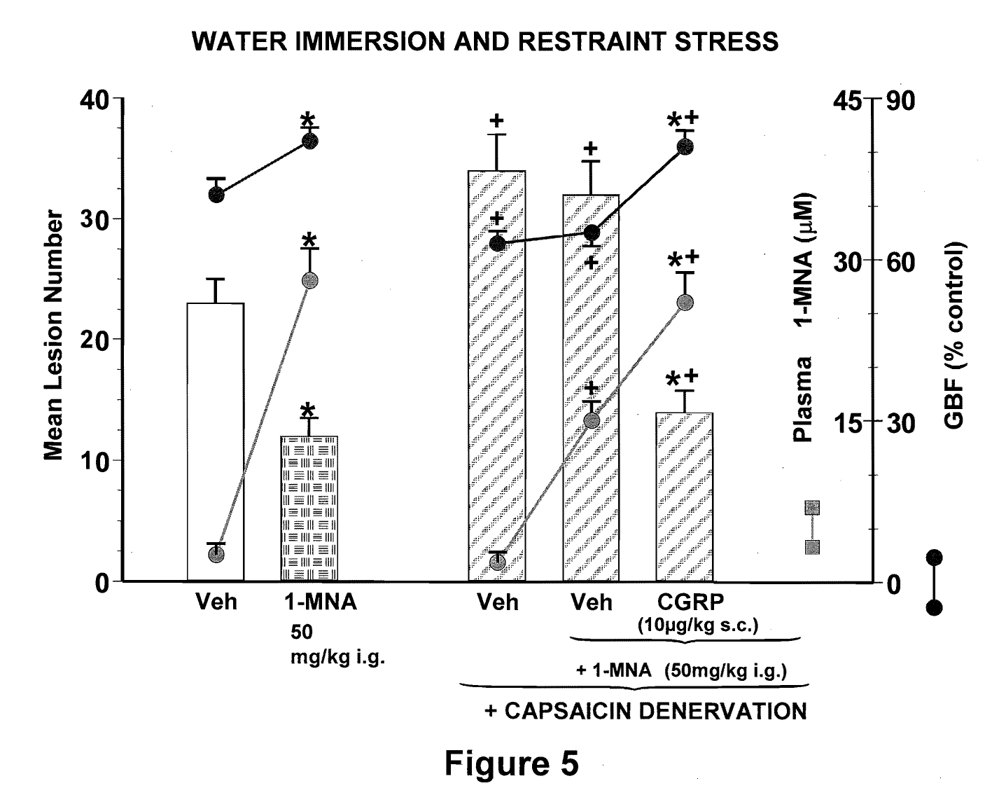

The next set of experiments was designed to assess the therapeutic activity of 1-MNA on gastric lesions with respect to capsaicin-induced denervation in preconditioned gastric mucosa. Capsaicin responsive sensory nerves are known to release NO and various vasodilatatory neuropeptides such as CGRP. The results demonstrate that capsaicin denervation was able to significantly inhibit the protection induced by 1-MNA with respect to gastric lesions. However, the addition of CGRP counteracted the deleterious effects of capsaicin-induced denervation (FIG. 5). Without wishing to be bound by any particular theory, it is believed that 1-NMA protects the gastric mucosa against injury induced by stress, and that the gastroprotective effect of 1-MNA is related to stimulation of endogenous CGRP and NO.

### Capsazepine

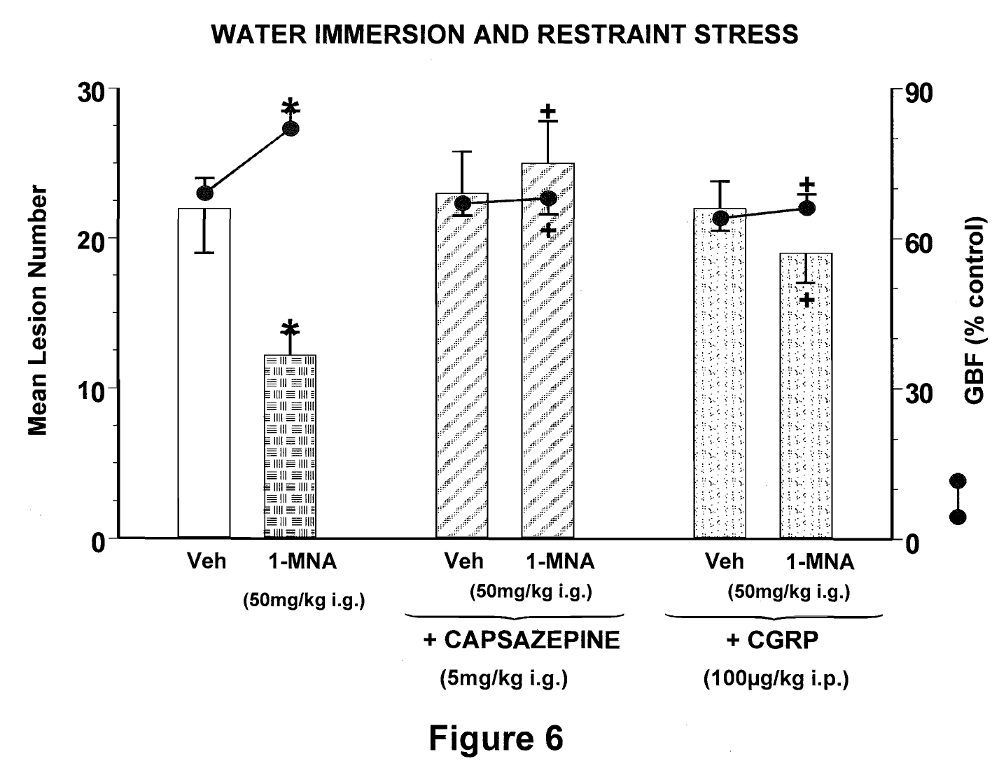

The next set of experiments was designed to assess the protective effects of 1-MNA on gastric mucosa injury in the presence of capsazepine (a competitive vanilloid receptor antagonist). In the WRS-induced ulceration model, it was observed that capsazepine reduced the protective property of 1-MNA against gastric mucosa injury as measured by mean lesion number and gastric blood flow. The addition of CGRP was able to counter the deleterious effects of casazepine (FIG. 6). Without wishing to be bound by any particular theory, it is believed that 1-MNA protects the gastric mucosa against injury induced by stress, and that the gastroprotective effect of 1-MNA is related to stimulation of endogenous CGRP released via activation of the vanilloid receptor.

The results presented herein demonstrate that gastroprotection and the accompanying rise in the GBF induced by 1-MNA were significantly attenuated by non-selective (indomethacin) and selective COX-1 (SC-560) and COX-2 (rofecoxib) inhibitors, as well as by capsaicin-induced denervation and capsazepine, which are known to be related to release of NO and other various vasodilatatory neuropeptides such as CGRP.

### Diabetes

The next set of experiments was designed to assess the therapeutic effects of 1-MNA in a combined diabetic and stress-induced gastric damage model.

Insulin-dependent diabetes exerts various influences on gastric functions, such as acid secretion and gastric emptying. It has observed that aggravation of gastric mucosal ulcerogenic responses occurs in diabetic rats. Furthermore, deleterious influence of diabetes on the healing of gastric mucosal lesions has been observed in diabetic rats.

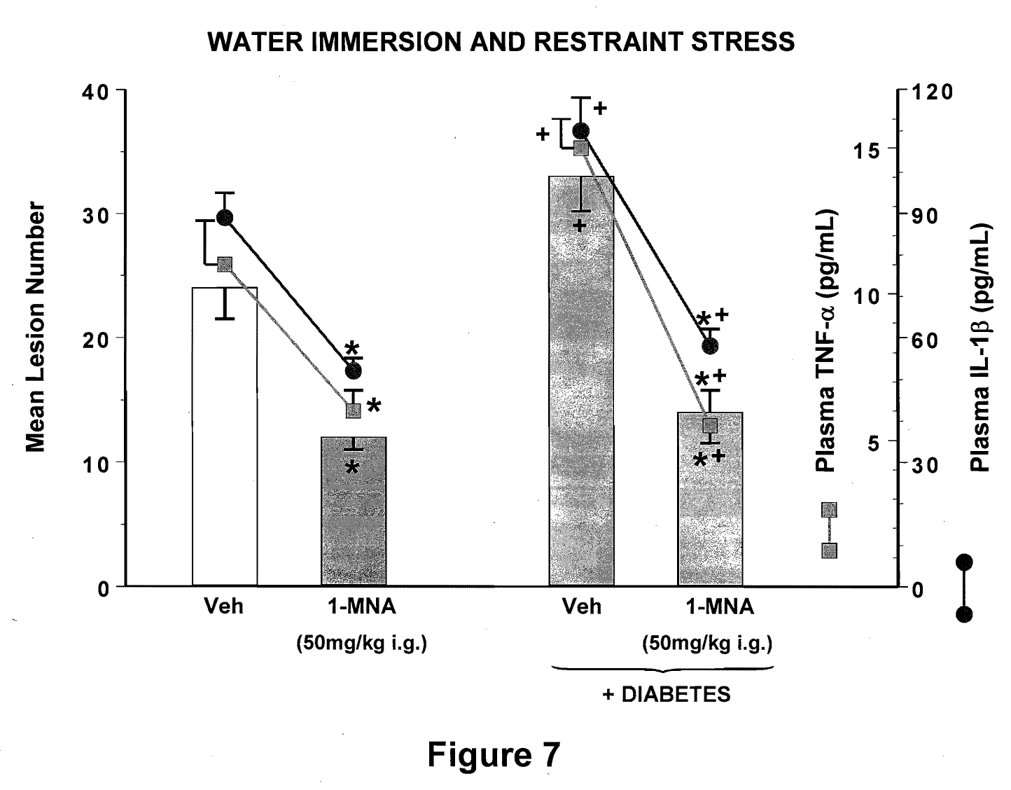

The results presented herein demonstrate that 1-MNA was able to reduce mean lesion number and decrease both plasma levels of IL-1β and TNFα. Thus, 1-MNA was shown to have a therapeutic effect in the combined diabetes and stress-induced gastric damage model (FIG. 7).

### Example 4

### Evaluation of 1-MNA on Gastric Lesions

### Ethanol Induced Lesion Model

The following experiments were designed to compare the effects of pretreatment with vehicle and 1-MNA or NA applied on ethanol-induced gastric lesions.

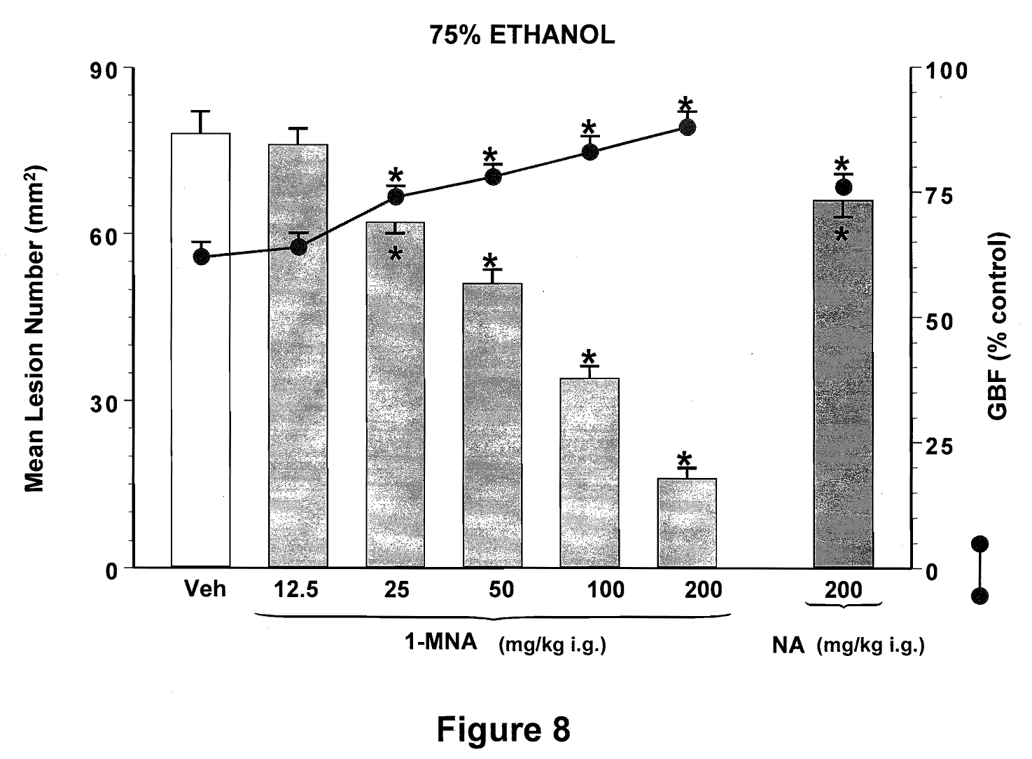

Pretreatment with 1-MNA resulted in decreased lesion number and increased gastric blood flow in ethanol-induced gastric lesion animal models. A comparison of the therapeutic effects of 1-MNA versus NA in the ethanol model demonstrated that 1-MNA was able to reduce lesion number and increase gastric blood flow more effectively than NA (FIG. 8).

### Cox Inhibitors

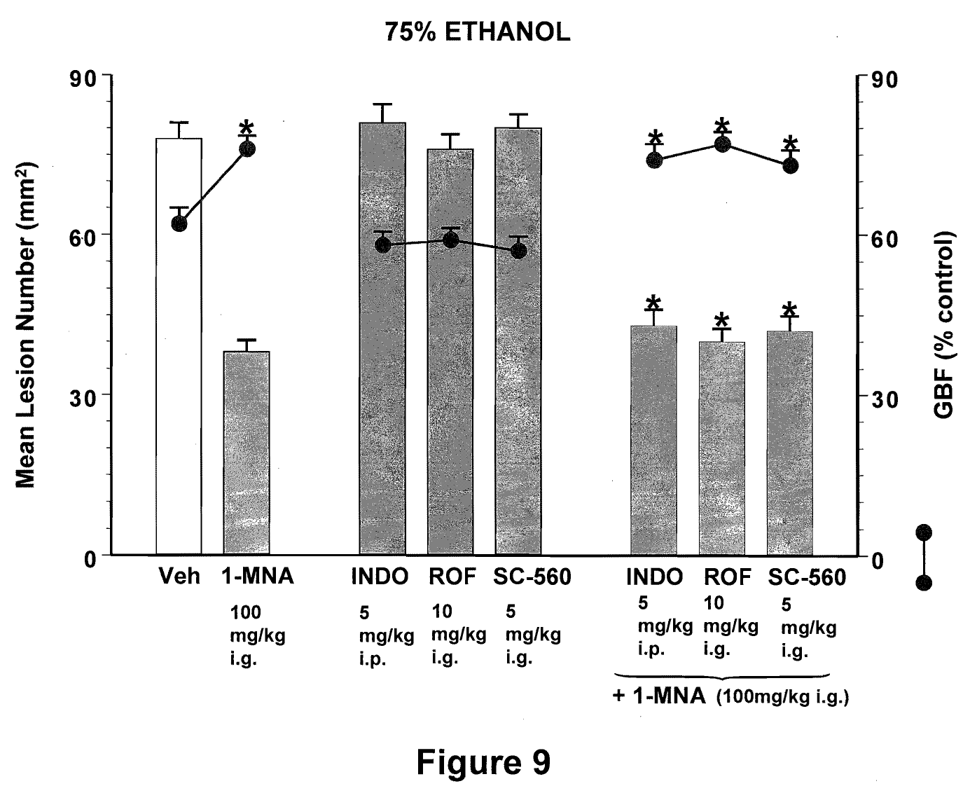

The therapeutic effect of 1-MNA in reducing gastric lesions was further assessed by administering vehicle or 1-MNA into animals with or without pretreatment with indomethacin (5 mg/kg i.p.), SC-560 (5 mg/kg i.g.) or rofecoxib (10 mg/kg i.g.). It was observed that pretreatment with either indomethacin, SC-560, or rofecoxib in the absence of 1-MNA increased the number of lesions compared to the number observed without treatment of indomethacin, SC-560, or rofecoxib in the ethanol-induced gastric lesion model. However, the mean lesion number in animals pretreated with either indomethacin, SC-560, or rofecoxib was significantly reduced in the presence of 1-MNA (FIG. 9). The addition of 1-MNA was able to counteract the deleterious effects of indomethacin, SC-560, or rofecoxib in the ethanol-induced gastric lesion model.

### Example 5

### Evaluation of 1-MNA on Gastric Lesions

### Acidified ASA Model

The following experiments were designed to compare the effects of pretreatment with vehicle and 1-MNA or NA applied on ASA-induced gastric lesions.

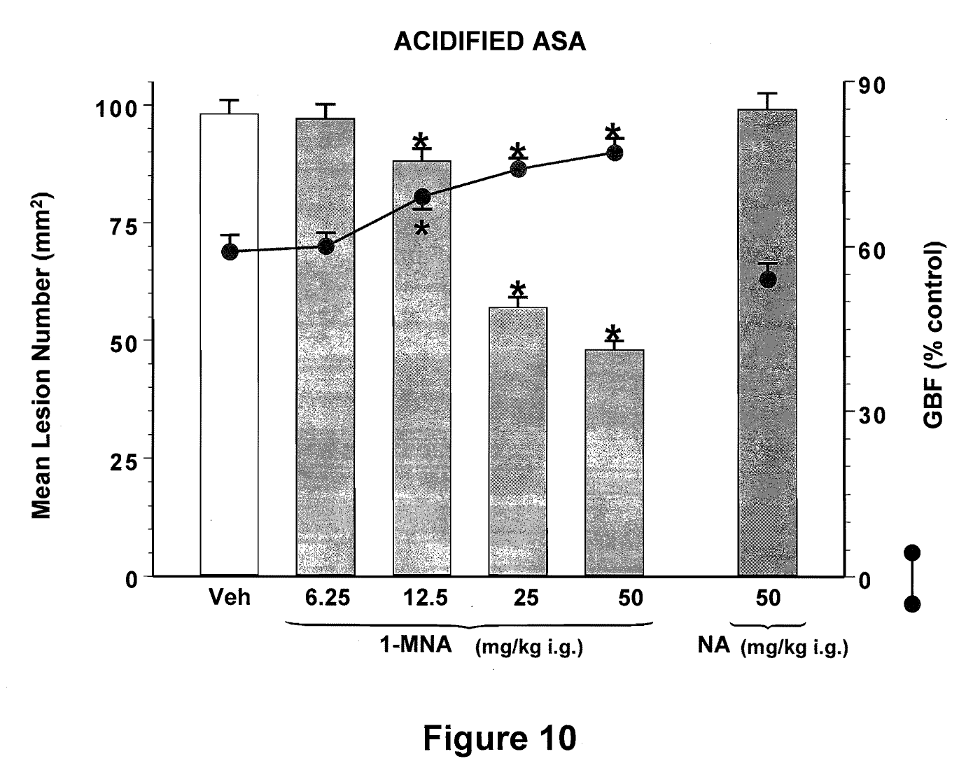

Pretreatment with 1-MNA resulted in decreased lesion number and increased gastric blood flow in ASA-induced gastric lesion animal models. A comparison of the therapeutic effects of 1-MNA versus NA in the ethanol model demonstrated that 1-MNA was able to reduce lesion number and increase gastric blood flow more effectively than NA (FIG. 10).

The next set of experiments were designed to measure myeloperoxidase (MPO), malondialdehyde (MDA) and 4-hydroxynonenal (4-HNE), and superoxide dismutase (SOD) activities in rat stomach exposed to acidified ASA as indicators of the prognosis of gastric mucosa injury. To evaluate the inflammatory changes, myeloperoxidase (MPO) activity in the gastric mucosa was measured. MPO activity was measured by enzyme assay. MDA and 4-HNE in tissue are accepted as major products of lipid peroxidation and thus are considered as indicators of mucosa injury by reactive oxygen species (ROS). Organisms have several enzymatic systems that scavenge ROS and prevent their destructive action. The major antioxidative enzyme is superoxide dismutase (SOD).

It has been demonstrated that administration of ASA into a mammal resulted in appearance of acute gastric lesions accompanied by a significant decrease of GBF. Also observed was a significant increase of mucosal levels of MDA and 4-HNE, and accompanied by a decrease of SOD activity. Pretreatment with NO-donors (e.g., SIN-1, SNAP, nitroglycerin, NO-ASA) resulted in reduction in gastric lesion number, increment of GBF, decrease of MDA and 4-HNE tissue level, and increase of SOD activity. Suppression of ROS plays an important role in the action of NO-donors on healing of acute gastric lesions. NO-donors caused an attenuation of lipid peroxidation as documented by a decrease of MDA and 4-HNE levels and enhancement of antioxidative properties as evidenced by an increase of SOD activity.

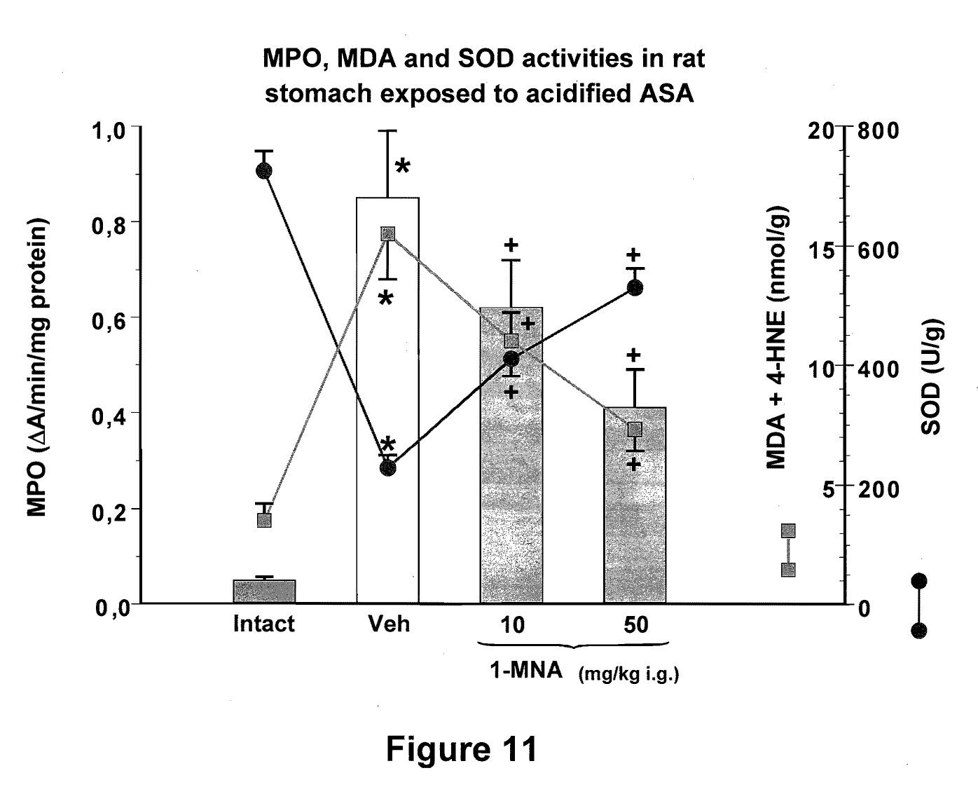

The administration of 1-MNA in the ASA-induced gastric lesion model contributed to lowering the levels of MPO, increasing the levels of SOD, and decreasing the levels of MDA and 4-HNE (FIG. 11).

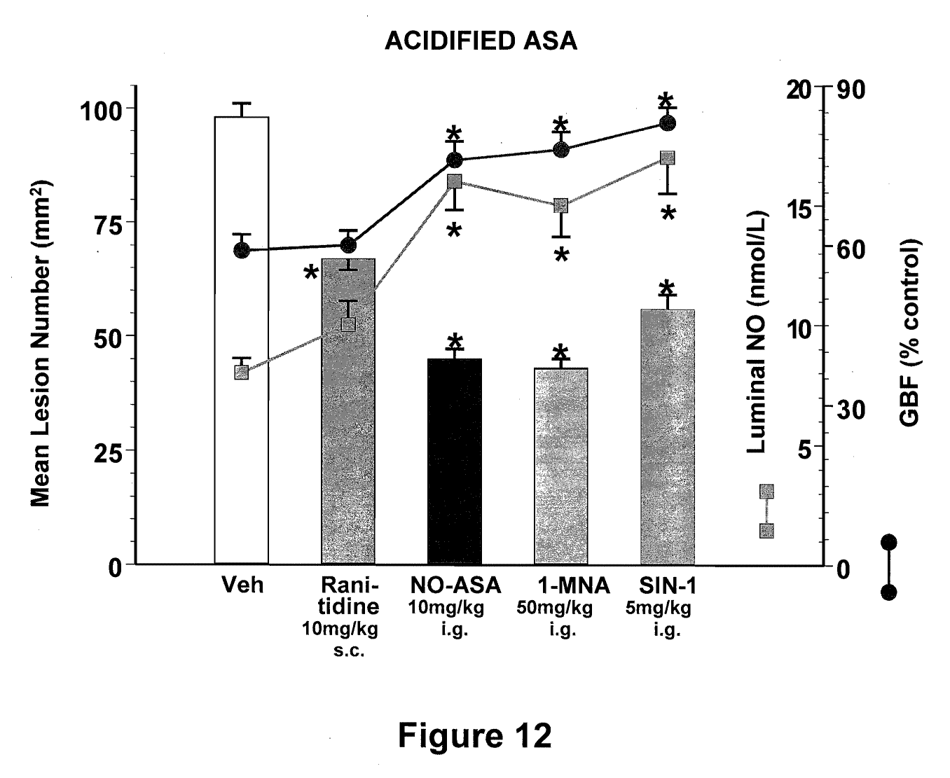

The next set of experiments were designed to compare the protective properties of 1-MNA with compounds observed to have protective properties on gastric lesions such as ranitidine (histamine H2 receptor antagonist), NO-ASA (NO-releasing aspirin), and SIN-1 (3-morpholino-sydnonymide; a donor of nitric oxide). The results presented herein demonstrate that 1-MNA was equally effective in reducing mean lesion number, increasing luminal NO, and increasing GBF (FIG. 12). Without wishing to be bound by any particular theory, NO is released in large quantities in the mucosa and contributes to the reduction of the area and number of gastric lesions.

### Example 6

### Evaluation of 1-MNA on Gastric Lesions

### Ischemia-Reperfusion (I/R)

The following experiments were designed to compare the effect of pretreatment with vehicle and 1-MNA on I/R-induced gastric lesions.

NO plays an important regulatory role in maintaining gastric mucosal integrity. NO is considered to have a gastroprotective factor, because when released in large quantities in the mucosa it contributes to the reduction of the area and number of gastric lesions. It has been shown that both endogenous NO released by capsaicin or NO originating from L-arginine, a substrate for NO-synthase (NOS), exert gastroprotective activity, mainly due to hyperemia and the maintenance of blood flow in stressed gastric mucosa. The contribution of NO with respect to cytoprotection was based on the finding that NG-nitro-L-arginine (L-NNA; inhibitor of NO-synthase) reversed the effect of NO. Furthermore, concurrent treatment with L-arginine, a substrate for NO-synthase or co-administered with L-NNA counteracted the inhibitory effect of L-NNA and increased GBF in gastric lesions. Alternatively, D-arginine, which is not a substrate for NO-synthase, had no significant effects on counteracting the inhibitory effect of L-NNA.

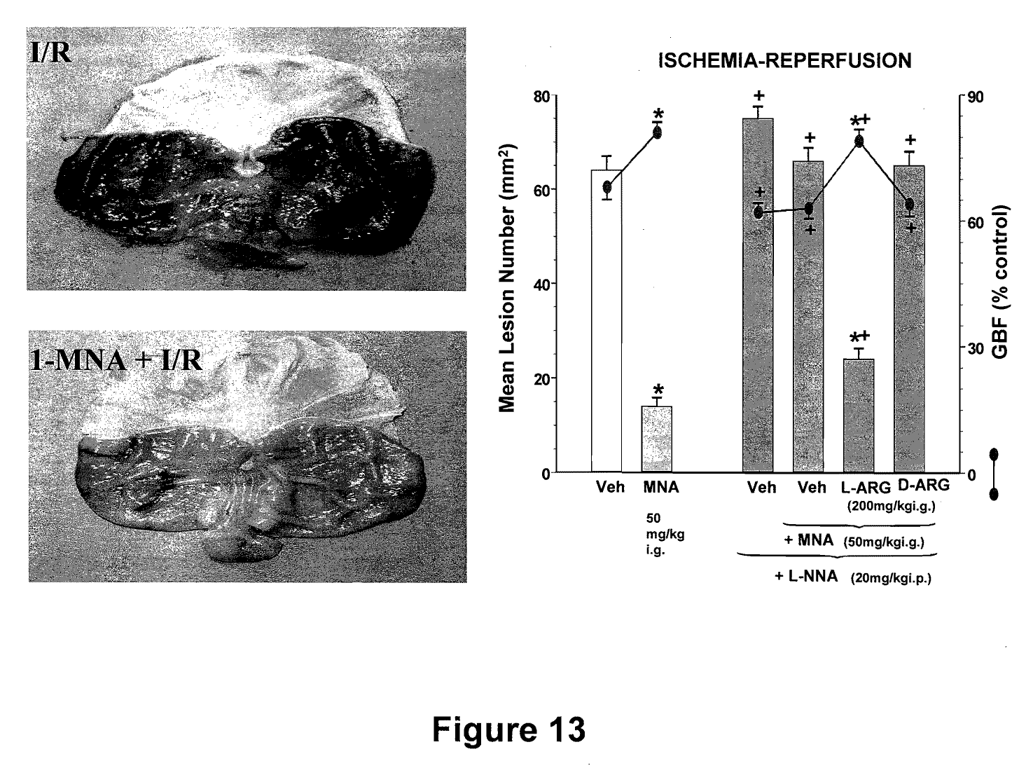

The results presented herein demonstrate that pretreatment with 1-MNA resulted in decreased lesion number and increased gastric blood flow in I/R-induced gastric lesion animal models. Administration of L-NNA was observed to reverse the therapeutic effects of 1-MNA on gastric lesions (FIG. 13).

### Example 7

### 1-MNA on Acetic Acid Ulcer

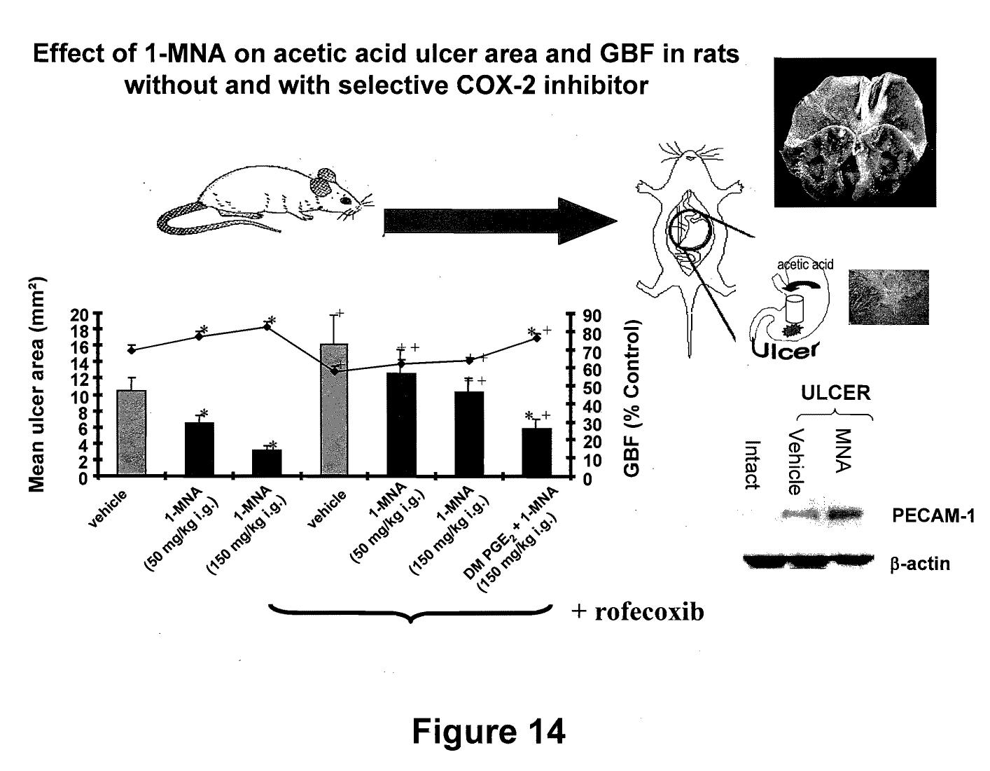

The following experiments were designed to assess the effects of 1-MNA on acetic acid ulcer area and GBF in rats administered with and without a selective COX-2 inhibitor (e.g., rofecoxib). The results demonstrate that administration of 1-MNA to a rat having a disorder associated with an ulcer served to reduce the ulcer area in the rat as well as to increase the GBF in the ulcer area. Furthermore, it was observed that 1-MNA was able to reduce the ulcer area in rats given rofecoxib as well as increase the GBF in the ulcer area. The combination of 1-MNA with DM PGE₂ in rats given rofecoxib served to reduce the ulcer area at a higher level in the rat as well as increase the GBF in the ulcer area as compared to administration of 1-MNA alone (FIG. 14).

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of treating a gastrointestinal disorder in a subject in need thereof, the method comprising administering to the subject a pharmaceutical composition comprising a compound of formula (I): 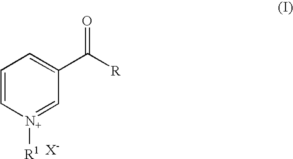
wherein
R is the group NR²R³ or the group OR⁴;
R¹ is methyl;
R² and R⁴ each independently is hydrogen or C₁₋₄alkyl;
R³ is hydrogen, C₁₋₄alkyl or CH₂OH; and
X⁻ is a physiologically suitable counter-anion,
with the proviso that said gastrointestinal disorder is not gastric ulcer or duodenal ulcer.

2. The method of claim 1, wherein R is the group NR²R³.

3. The method of claim 1, wherein R² is methyl or hydrogen.

4. The method of claim 1, wherein R³ is CH₂OH or hydrogen.

5. The method of claim 1, wherein R is the group OR⁴, and further wherein R⁴ is C₁₋₄ alkyl.

6. The method of claim 1, wherein R⁴ is propyl or ethyl.

7. The method of claim 1, wherein the compound of formula (I) is selected from a 1-methylnicotinamide salt or a 1-methyl-N′-hydroxymethylnicotinamide salt.

8. The method of claim 1, wherein the compound of formula (I) is selected from a 1-methylnicotinic acid ethyl ester salt or a 1-methylnicotinic acid propyl ester salt.

9. The method of claim 1, wherein the compound of formula (I) is selected from a 1-methylnicotinic acid salt.

10. The method of claim 1, wherein the compound of formula (I) is selected from 1-methylnicotinamide chloride, 1-methylnicotinamide citrate, 1-methylnicotinamide lactate, 1-methyl-N′-hydroxymethylnicotinamide chloride, 1-methylnicotinic acid chloride, 1-methylnicotinic acid ethyl ester chloride or 1-methylnicotinic acid propyl ester chloride.

11. The method of claim 1, wherein the gastrointestinal disorder is associated with the development and progress of gastric mucosal lesion.

12. The method of claim 1, wherein the gastrointestinal disorder is associated with irritant-, ethanol-, stress-, or ischemia/reperfusion-induced gastric lesions.

13. The method of claim 1, wherein the gastrointestinal disorder is associated with undue gastric acid secretion, peptic ulcer, gastric mucosal damage, stress ulcers, gastric hyperacidity, dyspepsia, gastroparesis, Zollinger-Ellison syndrome, gastroesophageal reflux disease, short-bowel (anastomosis) syndrome, hypersecretory states associated with systemic mastocytosis or basophilic leukemia and hyperhistaminemia, or a bleeding peptic ulcer.

14. The method of claim 1, wherein the compound of formula (I) is administered orally, nasally, rectally, intravaginally, parenterally, buccally, sublingually, intragastrically or topically.

15. The method of claim 1, wherein the compound of formula (I) is administered orally.

16. The method of claim 1, wherein the compound of formula (I) is formulated using one or more pharmaceutically acceptable excipients selected from the group consisting of starch, sugar, cellulose, diluent, granulating agent, lubricant, binder, disintegrating agent, wetting agent, emulsifier, coloring agent, release agent, coating agent, sweetening agent, flavoring agent, perfuming agent, preservative, antioxidant, plasticizer, gelling agent, thickener, hardener, setting agent, suspending agent, surfactant, humectant, carrier, stabilizer, and any combinations thereof.

17. The method of claim 1, wherein the subject is a mammal.

18. The method of claim 1, wherein the subject is a human.

19. The method of claim 1, wherein the composition further comprises an anti-ulcer composition.

20. The method of claim 19, wherein the anti-ulcer composition is selected from the group consisting of an antibacterial agent, an alginate, a prokinetic agent, an H2-receptor antagonist, a proton pump inhibitor (PPI), a promotility agent, an antacid, sucralfate, heparin, and any combination thereof.
